(19) United States
(12) Patent Application Publication
(10) Pub. No.: US 2010/0104656 A1
(43) Pub. Date: Apr. 29, 2010

(54) Meltable Binder for Melt Granulation and/or Pelletization

(76) Inventors: Paul Wan Sia Heng, Singapore (SG); Wai See Cheong, Singapore (SG); Zhi Hui Loh, Singapore (SG); Dipan B. Ray, Old Bridge, NJ (US); Rama Haldar, Randolph, NJ (US); Yolande Anthony, Singapore (SG)

Correspondence Address: INTERNATIONAL SPECIALTY PRODUCTS;WILLIAM J. DAVIS, ESQ., 1361 ALPS ROAD, WAYNE, NJ, 07470, US

(21) Appl. No.: 12/531,329

(22) PCT Filed: Mar. 19, 2008
(86) PCT No.: PCT/US08/57477

§ 371 Date: Jan. 8, 2010

Related U.S. Application Data
(60) Provisional application No. 60/918,996, filed on Mar. 20, 2007.

(51) Int. Cl. A61K 9/14; A61K 8/72; A61K 47/32

(52) U.S. Cl. 424/501; 523/105; 514/772.5; 514/772.6

(57) ABSTRACT

A low melting binder composition for preparation of pharmaceutical tablets and/or granules which comprises a polymer and a hydrophobic meltable binder.

### BACKGROUND OF THE INVENTION

1. Field of the Invention

This invention relates to melt granulation and pelletization, and, more particularly to a binder composition comprising a polymer and a hydrophobic meltable co-binder useful in the production of pharmaceutical granules and/or pellets without water or organic solvent.

2. Description of the Prior Art

Melt granulation and melt pelletization in high shear mixers are agglomeration processes that have gathered increasing interest in the pharmaceutical industry for the purpose of utilizing a molten liquid as a binder. With meltable binders, there is no need for aqueous or organic solvents. Accordingly, no drying step is required which shortens the total processing time and lowers the cost of operation. Furthermore, water-sensitive materials such as effervescent excipients and hygroscopic drugs can be processed using this non-aqueous method of granulation and/or pelletization.

Melt agglomeration processes often are carried out in a high shear mixer where the high shearing forces of the impeller is utilized to achieve melting of a binder efficiently within a reasonably short time period and to distribute the molten liquid homogenously and as highly spherical agglomerates.

The major challenges in this technology are that the melt process is highly sensitive both to processing and equipment parameters and to physical properties of the meltable binders used, particularly to the vast differences in their physicochemical properties such as viscosity, surface tension and contact angle over the bulk powder. Previously meltable binders have included hydrophilic polyethylene glycols (PEGs) and poloxamers, and hydrophobic fatty acids, fatty alcohols, waxes, hydrogenated vegetable oils and glycerides. The hydrophobic meltable binders generally present problems in the process of melt agglomeration with a high shear mixer because these binders usually have low viscosity values, e.g. less than 50 mPa·s within a temperature range of 60° to 90° C., as well as a relatively high degree of electrostatic charging. Thus melt granules and/or pellets prepared using such hydrophobic meltable binders may have a tendency to break under the impact of the high shearing forces of high speed mixers; furthermore these binders have a rather low binding capability due to their low viscosities, thus forming products with a relatively wide size distribution and with low product yields.

Accordingly, it is an object of this invention to provide binders and improved melt agglomeration employing high shear mixes.

Another object therein is to provide a meltable binder composition which incorporates a polymer and hydrophobic meltable binder, either in the form of (a) physically mixed powder mixtures or (b) thermoplastic compositions obtained by (i) milling the solidified molten mixtures of the component or (ii) spraying the molten mixtures into a cool chamber wherein congealed fine particles are obtained.

Still another object is to provide a binder composition including a copolymer and hydrophobic meltable binder.

Yet another object is to provide such a binder composition which provides improved binding, product yield, compressibility and morphology characteristics.

Another object of this invention relates to a process of preparation of a thermoplastic composition comprising a copolymer and a hydrophobic meltable binder through spray-congealing whereby a homogeneous, free-flowing powder of the thermoplastic composition of the copolymer and the meltable binder is obtained which possesses an increased viscous binding strength.

### SUMMARY OF THE INVENTION

What is described herein is a low melting binder composition suitable for the preparation of drug tablets and/or granules which comprises a polymer and a hydrophobic meltable binder material.

A feature of the invention is that: (a) the physical mixture or thermoplastic composition of the polymer and the hydrophobic binder in particle form, and (b) the granulation and/or pelletization step is performed by mechanically working the mixture in a high shear mixer, under the input of a sufficient amount of energy for the binder to melt and granulation and/or pelletization to take place.

### IN THE DRAWINGS

FIG. 1 shows the physical appearance of a thermoplastic composition of 10% w/w PVP/VA S630 and cetyl alcohol prepared by (a) milling method and (b) spray-congealing method.

FIG. 2 is a plot of viscosity vs. conc. of S630 in a molten liquid composition of S630 and cetyl alcohol.

FIG. 3 is a plot of tensile strength vs. binder concentration for melt granules obtained with a thermoplastic composition of PVP/VA S630 and cetyl alcohol (with 15% w/w of PVP/VA S630) as compared to cetyl alcohol alone.

FIG. 4 shows the physical appearance of melt granules obtained with a thermoplastic composition of PVP/VA S630 and cetyl alcohol (with 15% w/w of PVP/VA S630) compared to cetyl alcohol alone.

FIG. 5 is a plot of tensile strength vs. concentration of S630 for melt granules obtained with a thermoplastic composition of PVP/VA S630 and cetyl alcohol; granule size fraction 1.0-1.4 mm.

FIG. 6 is a plot of crushing strength of tablets prepared from melt granules using a thermoplastic composition of PVP/VA S630 and cetyl alcohol as meltable binder as a function of PVP/VA S630 concentration (as a weight percentage to the total weight of the composition.

### DETAILED DESCRIPTION OF THE INVENTION

In accordance with the objects of the present invention, there is provided herein an effective meltable binder composition which is capable of producing melt granules and pellets with desirable features. The composition of the invention comprises:

a) various concentrations, generally around 1 to 75, preferably 3 to 60 and most preferably 10 to 50 wt. % of a polymer, preferably selected from vinyl pyrrolidone/vinyl acetate copolymers, with PVP/VA S630 (ISP Corp.) being the most preferable. PVP/VA S630 is a 60:40 wt/wt random amorphous copolymer of N-vinyl-2-pyrrolidone and vinyl acetate with a relatively low glass transition temperature, high plasticity and viscosity which provide effective binding strength to agglomerates, and good flow properties for even distribution in a high shear mixer bowl and a relatively low hygroscopicity which enables moisture sensitive materials to be used, and,

b) 25-99, preferably 40-97 and most preferably 50 to 90 wt. % of hydrophobic meltable binder, of a wax-like binder substance preferably including a long chain fatty alcohol, fatty acid or ester thereof, with cetyl alcohol being the most preferable. The binder suitably is in particle form with a melting point above 40° C.

The composition suitably is used at concentrations of 5-40 wt. %, preferably 10-30 wt. %, with respect to active ingredients and/or fillers in the formulation.

The composition can be used in melt granulation and/or melt pelletization as a binder/co-binder in the form of either (a) a physical mixture where the copolymer is to be added with the hydrophobic meltable binder together with other fillers/drugs, (b) a thermoplastic composition where the copolymer is melted together with the hydrophobic meltable binder and subsequently a fine powder of the composition is obtained by milling down the congealed composition; or (c) a thermoplastic composition where the copolymer is melted together with the hydrophobic meltable binder and subsequently sprayed into a cooled chamber to obtain a free-flowing congealed powder of narrow size distribution that possesses increased viscous binding strength for the melt granules and/or pellets.

To prepare the thermoplastic composition of the copolymer and the hydrophobic meltable binder via method (b) above, a mixture of these substances is heated to above their melting temperature to obtain a homogeneous molten liquid. The molten liquid then is subsequently cooled down to obtain a congealed mass. The mass thereafter is further milled down to a fine powder by passing it through a mill such as a Fitzpatrick mill. A powder of size fraction less than 710 μm, and, preferably, less than 350 μm, is obtained and used for melt granulation and/or pelletization.

To prepare the thermoplastic composition of the copolymer and the hydrophobic meltable binder via method (c) above, molten liquid of a mixture of 10-20% w/w of S630 in the meltable binder in the most preferable composition, is obtained by heating it to above its melting temperatures. Then the composition is atomized as droplets in a cooled chamber of a spray congealer maintained at a temperature below the melting range of the substances, preferably, in the range of 12-20° C. The spraying speed and atomizing pressure is adjusted to obtain micropellets of the thermoplastic composition within a desired size range.

A high speed mixer suitable for the production of melt granules and/or pellets is described in U.S. Pat. Nos. 5,030,400; 5,807,583 and 6,162,467, where the copolymer and hydrophobic meltable binder either in the form of a physical mixture or thermoplastic composition are added in a formulation with other suitable fillers and/or drugs, and subjected to mixing. The melting of the composition achieved either by external equipment such as a water jacket and/or internal melting of the frictional heat generated from the interparticulate high-speed movement within the processing chamber of the said high speed mixer. With an appropriate mixing speed and time, melt granules and/or pellets can be obtained.

The thermoplastic composition of PVP/VA S630 and a meltable binder, most preferably cetyl alcohol, may be prepared by melting the two components in a suitable container above their melting temperatures, preferably at 60-80° C., until a clear yellowish molten liquid is obtained. The molten liquid then can be further processed into fine powders by either of two routes:

(a) congeal the molten liquid at room temperature and mill the congealed mass to a fine powder using a mill such as a Fitzpatrick mill; or

(b) spray the molten liquid into a cool chamber in fine droplets by applying an atomizing air pressure, preferably between 0.2 to 1.5 bar; whereupon the liquid congeals within the cooled chamber to form a fine powder.

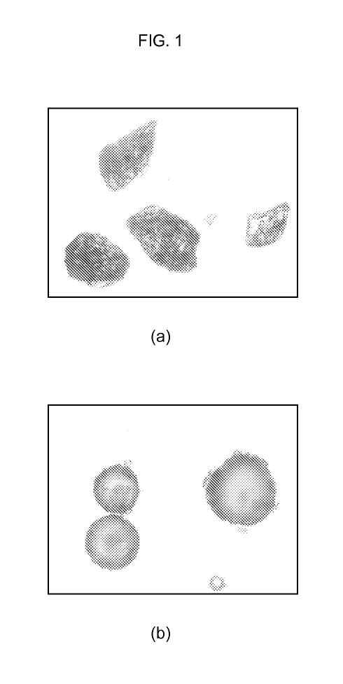

For method (a), a concentration of PVP/VA S630 as high as 50% w/w is suitable; while for method (b), a concentration of PVP/VA S630 between 5-20% w/w, is preferable thereby providing highly spherical fine powders (FIG. 1b). Less spherical and a more irregularly shaped thermoplastic composition of PVP/VA S630 is obtained through method (a) (FIG. 1a).

A composition of PVP/VA S630 and a meltable binder is used to prepare melt granules and/or pellets in a high shear mixer by first premixing all the powders in the processing chamber of a high shear mixer at a temperature below the melting point of the meltable binder. Subsequently, the chamber temperature is raised by applying an external heating element such as a water-jacket or by utilizing the high speed of an impeller to induce frictional heat onto the mixing powders within the chamber, causing them to reach the melting point of the meltable binder. The molten form of the meltable binder then can bind the solid powders under agitation to form granules and/or pellets. An alternative method is to add the thermoplastic composition of PVP/VA S630 and meltable binder, in the form of a molten liquid, by preheating the said composition, and adding the solid powders into the processing chamber of a high shear mixer. The molten liquid of the resultant composition then can bind the solid powders to form granules and/or pellets under agitation.

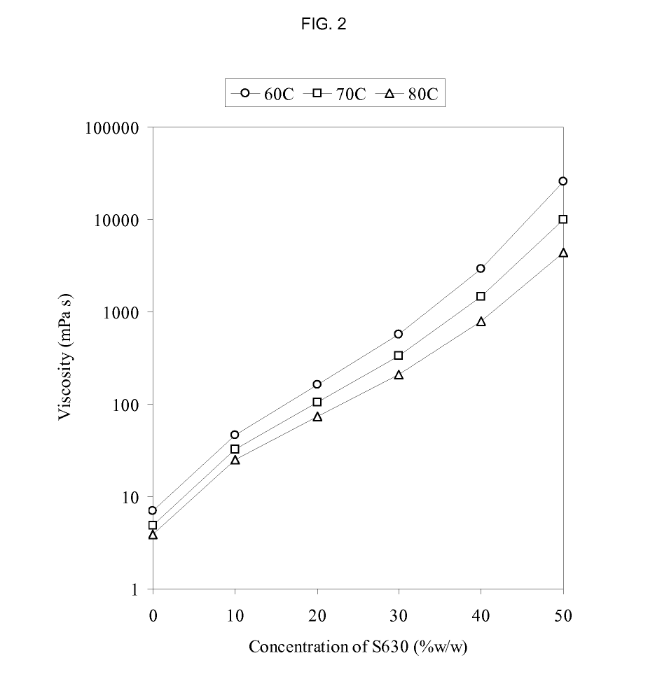

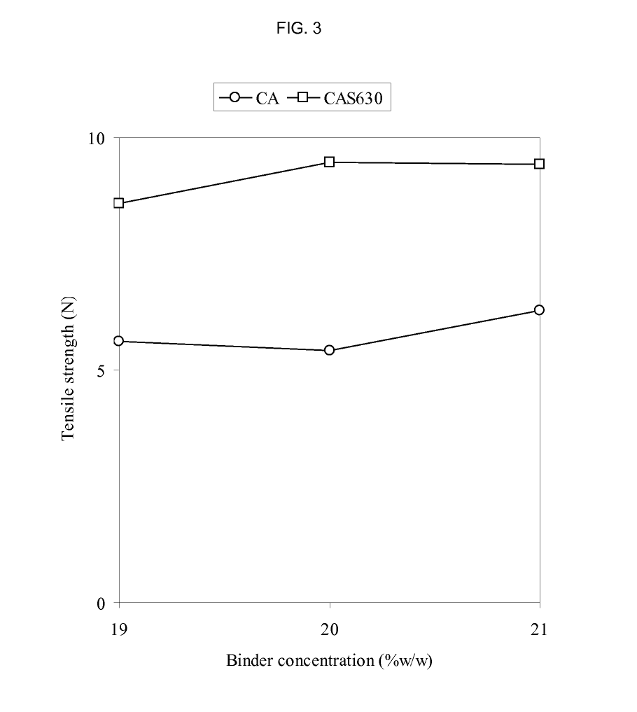

The incorporation of PVP/VA S630 into a meltable binder increases the viscosity of the latter (FIG. 2). The increased viscosity of the meltable binder will bind the solid particles more effectively due to increased viscous forces of the binding liquid during the granulation process, thus reducing the breakage tendency frequently associated with the hydrophobic binders. The resultlant effect is to narrow the size distribution of the melt granules (FIG. 3) and improve the product yield.

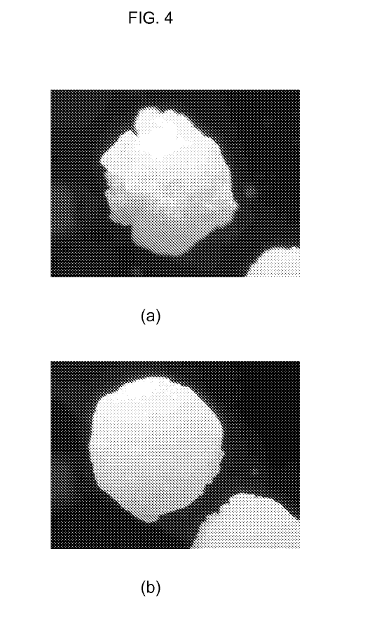

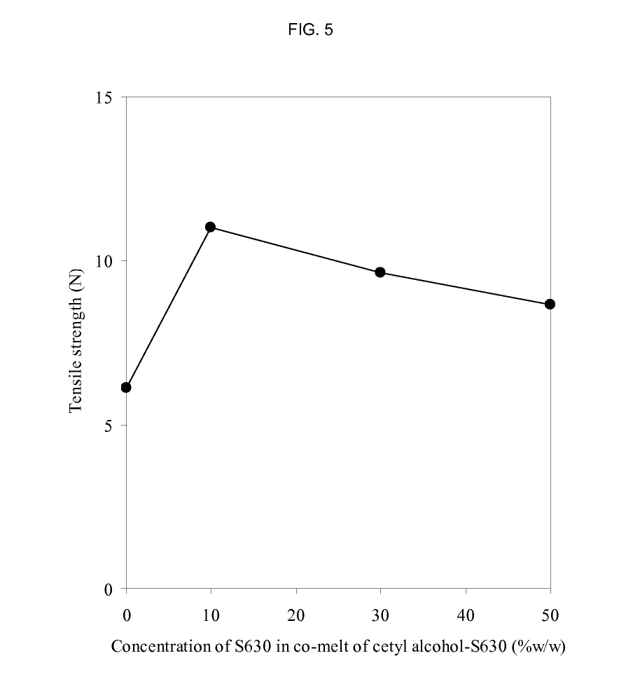

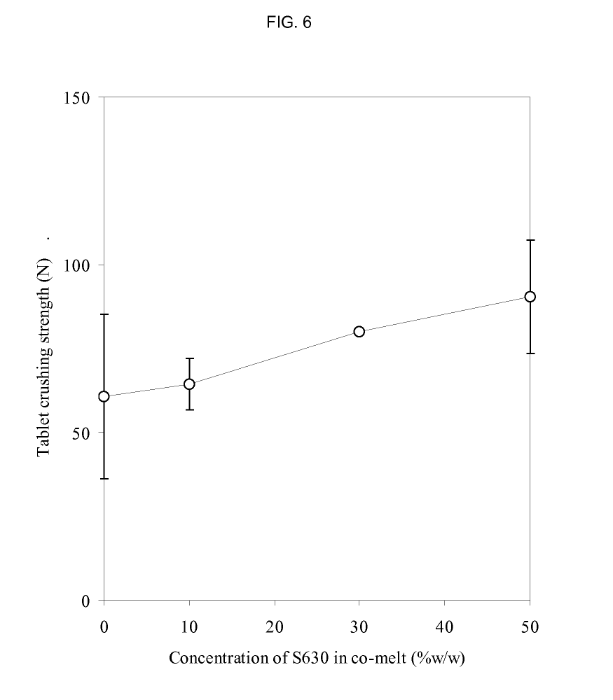

Since the melt granules are stronger in the presence of PVP/VA S630 (FIG. 4), breakage is reduced and the melt granules are more rounded as compared to those without PVP/VA S630 (FIG. 5). A high PVP/VA S630 concentration (more than 30% w/w) increases the viscosity of the meltable binder to about a 3-4 order of magnitude of the viscosity of the meltable binder without the incorporation of PVP/VA S630. This results in weaker melt granules (FIG. 6) because with a very high viscosity in the region of 1000-10000 mPa s, consolidation of the melt granules can be compromised as interparticulate movement becomes difficult. Nonetheless, the use of such melt granules for compression into tablets still results in strong tablets (FIG. 7) which is particularly useful for the preparation of the tablets containing powders of an elastic nature, e.g., the drugs paracetamol and mefenamic acid.

The invention will now be described with reference to the following non-limiting examples.

### EXAMPLE 1

A mixture of 800 g of the drug metformin HCl and 160 g of a composition of PVP/VA S630-cetyl alcohol (with 50% w/w PVP/VA S630) was used for granulation. The mixing speed and time upon melting of the meltable binder was 1000 rpm for 1 min, followed by 500 rpm for 5 min. Jacket temperature: 55° C. (external heating). Sieve analysis as follows:

TABLE 1

| Diameter (μm) | Wt. % | Cumulative % |
|---|---|---|
| 90 | 0.4 | 0.4 |
| 125 | 1.1 | 1.5 |
| 180 | 2.4 | 3.9 |
| 250 | 6.5 | 10.4 |
| 355 | 26.3 | 36.7 |
| 500 | 27.9 | 64.6 |
| 710 | 13.9 | 78.5 |
| 1000 | 6.5 | 85.0 |
| 2000 | 6.3 | 91.3 |
| 2800 | 3.9 | 95.2 |
| 4000 | 4.8 | 100.0 |

### EXAMPLE 2

A mixture of 800 g of the drug paracetamol and 220 g of a composition of PVP/VA S630-cetyl alcohol (with 50% w/w PVP/VA S630) was used for granulation. The mixing speed and time upon melting of the meltable binder was 1200 rpm for 2 min, followed by 355 rpm for 5 min. Jacket temperature: 55° C. (external heating). Sieve analysis as follows:

TABLE 2

| Diameter (μm) | Wt. % | Cumulative % |
|---|---|---|
| 90 | 11.3 | 11.3 |
| 125 | 24.4 | 35.7 |
| 180 | 22.2 | 57.9 |
| 250 | 23.9 | 81.8 |
| 355 | 9.8 | 91.6 |
| 500 | 4.7 | 96.3 |
| 710 | 2.3 | 98.6 |
| 1000 | 1.0 | 99.6 |
| 2000 | 0.4 | 100 |
| 2800 | 0 | 100 |
| 4000 | 0 | 100 |

### EXAMPLE 3

A mixture of the drug 800 g of aspirin and 84 g of a composition of cetyl alcohol and 36 g of PVP/VA S630 was used for granulation. The mixing speed and time upon melting of the meltable binder was 1200 rpm for 2 min, followed by 400 rpm for 5 min. Jacket temperature: 55° C. (external heating). Sieve analysis as follows:

TABLE 3

| Diameter (μm) | Wt. % | Cumulative % |
|---|---|---|
| 90 | 0 | 0 |
| 125 | 0.1 | 0.1 |
| 180 | 0.3 | 0.4 |
| 250 | 1.6 | 2.0 |
| 355 | 4.4 | 6.4 |
| 500 | 12.4 | 18.8 |
| 710 | 23.5 | 42.3 |
| 1000 | 31.3 | 73.6 |
| 1400 | 17.1 | 90.7 |
| 2000 | 3.4 | 94.1 |
| 4000 | 5.9 | 100.0 |

### EXAMPLE 4

A mixture of 1000 g of lactose monohydrate and 200 g of PVP/VA S630-cetyl alcohol and cetyl alcohol alone was used for granulation. The mixing speed and time upon melting of the meltable binder was 1200 rpm for 5 min, followed by 400 rpm for 5 min. Jacket temperature: 55° C. (external heating). Sieve analysis as follows:

TABLE 4

| | PVP/VA S630-cetyl alcohol | | Cetyl alcohol only | |
|---|---|---|---|---|
| Diameter | | Cumulative | | Cumulative |
| (μm) | Wt. % | % | Wt. % | % |
| 90 | 1.0310781 | 1.031078 | 3.8776331 | 3.877633 |
| 125 | 0.5122967 | 1.543375 | 4.1497477 | 8.027381 |
| 180 | 1.2256211 | 2.768996 | 12.510469 | 20.53785 |
| 250 | 1.4071946 | 4.176191 | 10.292735 | 30.83058 |
| 355 | 0.8884132 | 5.064604 | 6.5579619 | 37.38855 |
| 500 | 4.7533349 | 9.817939 | 5.1633746 | 42.55192 |
| 710 | 3.5666223 | 13.38456 | 4.7688084 | 47.32073 |
| 1000 | 34.615691 | 48.00025 | 9.2518965 | 56.57263 |
| 1400 | 46.294758 | 94.29501 | 10.408384 | 66.98101 |
| 2000 | 5.1035123 | 99.39852 | 20.980036 | 87.96105 |
| 2800 | 0.0259391 | 99.42446 | 7.8028862 | 95.76393 |
| 4000 | 0.0778172 | 99.50228 | 2.1292968 | 97.89323 |
| 6000 | 0.4977216 | 100 | 2.1067716 | 100 |

### EXAMPLE 5

A mixture of 1000 g of lactose monohydrate and 200 g of PVP/VA S630-stearic acid and stearic acid alone was used for granulation. The mixing speed and time upon melting of the meltable binder was 1200 rpm for 5 min, followed by 400 rpm for 5 min. Jacket temperature: 55° C. (external heating). Sieve analysis as follows:

TABLE 5

| | PVP/VA S630-stearic acid | | Stearic acid only | |
|---|---|---|---|---|
| Diameter | | Cumulative | | Cumulative |
| (μm) | Wt. % | % | Wt. % | % |
| 90 | 1.4225701 | 1.42257 | 2.0580097 | 2.05801 |
| 125 | 1.2857845 | 2.708355 | 6.3928209 | 8.450831 |
| 180 | 2.8724974 | 5.580852 | 14.994071 | 23.4449 |
| 250 | 2.8109439 | 8.391796 | 9.6473479 | 33.09225 |
| 355 | 1.2789453 | 9.670741 | 4.3279739 | 37.42022 |
| 500 | 2.3595514 | 12.03029 | 2.8784787 | 40.2987 |
| 710 | 16.202253 | 28.23255 | 2.2973132 | 42.59602 |
| 1000 | 56.506127 | 84.73867 | 9.421719 | 52.01773 |
| 1400 | 13.979487 | 98.71816 | 24.702954 | 76.72069 |
| 2000 | 0.683928 | 99.40209 | 20.204049 | 96.92474 |
| 2800 | 0.0683928 | 99.47048 | 1.7434966 | 98.66823 |
| 4000 | 0.1162678 | 99.58675 | 0.7247476 | 99.39298 |
| 6000 | 0.4132511 | 100 | 0.6070185 | 100 |

What is claimed is:

1. A low melting binder composition for preparation of pharmaceutical tablets and/or granules which comprises a polymer and a hydrophobic meltable binder.

2. A composition according to claim 1 wherein the polymer is a copolymer of polyvinyl pyrrolidone and vinyl acetate.

3. A composition according to claim 2 wherein said copolymer is an amorphous, random copolymer in a wt/wt ratio of about 60:40.

4. A composition according to claim 1 wherein the hydrophobic meltable binder is a long chain fatty alcohol, fatty acid or ester thereof.

5. A composition according to claim 4 wherein said binder is a fatty alcohol.

6. A composition according to claim 5 wherein said fatty alcohol is cetyl alcohol.

7. A composition according to claim 2 which comprises 1 to 75 wt. % of said copolymer and the rest is said hydrophobic meltable binder.

8. A formulation including the composition of claim 1 and an active ingredient and/or filter in which the composition is present in an amount of 5-40 wt. % of the formulation.

9. A formulation according to claim 8 wherein said amount is 10-30 wt. %.

10. A powder of said composition of claim 1 or said formulation of claim 8 having a size fraction less than 710 μm.

11. A powder of claim 10 having a size fraction less than 350 μm.

12. A free-flowing composition according to claim 1 having a melting point of about 40-100° C.

13. A composition according to claim 1 having a viscosity of 1000 to 10,000 mPas.

14. A composition according to claim 1 having a tensile strength of about 10 N.

15. A tablet formed from the composition of claim 1 having a tablet crushing strength of 50 to 75 N.

16. A process of making the composition of claim 1 which comprises mixing the components, melting and milling to provide a powder thereof.
